(19) United States
(12) Patent Application Publication
(10) Pub. No.: US 2011/0070277 A1
(43) Pub. Date: Mar. 24, 2011

(54) Substrate Comprising A Lotion Composition Limiting the Adherence of Feces or Menses to the Skin

(76) Inventors: Victor Nicholas Vega, Cincinnati, OH (US); Linda Jean Bryson, Cincinnati, OH (US)

(21) Appl. No.: 12/885,735

(22) Filed: Sep. 20, 2010

Related U.S. Application Data
(60) Provisional application No. 61/243,645, filed on Sep. 18, 2009.

(51) Int. Cl. A61K 8/02; A61K 8/86; B05D 3/00; A61K 8/36; A61K 8/81; A61Q 19/00; G01N 25/20

(52) U.S. Cl. 424/402; 424/78.31; 427/2.31; 514/558; 514/723; 374/10

(57) ABSTRACT

A lotion composition applied on the body facing surface of an absorbent article such as a diaper, training pant, adult incontinence product, feminine hygiene product, improves the ease of removal of feces or menses after an absorbent article comprising the lotion composition has been used and removed from the wearer. The lotion composition may also suitably be applied on a wipe.

### CROSS REFERENCE TO RELATED APPLICATION

This application claims the benefit of U.S. Provisional Application No. 61/243,645, filed Sep. 18, 2009, which is herein incorporated by reference in its entirety.

### FIELD OF THE INVENTION

A lotion composition deposited on a substrate comprised by, or forming, or used for manufacturing of, an absorbent article such as a diaper, training pant, adult incontinence product or feminine hygiene product may be used for reducing the adherence of feces or menses to the human skin. The lotion composition deposited on the substrate comprised by, or forming, or used for manufacturing of, an absorbent article does not affect negatively the manufacturing process and imparts desirable fluid handling properties to the substrate. The lotion composition may also be desirably deposited on a substrate used as, or in a wipe.

### BACKGROUND OF THE INVENTION

Disposable absorbent products, such as diapers and sanitary napkins that have a topsheet comprising a lotion to deliver skin benefits to the skin of the wearer are known. In recent years the focus has been to deliver lotions to sanitary napkins and diapers that provide extra skin benefits, for example by addition of botanical ingredients or pharmaceutical ingredients to the lotions. Lotions of various types are known to provide various skin benefits, such as prevention or treatment of diaper rash. These lotions can be applied to the topsheet of absorbent articles, and can be transferred to the skin of the wearer during use. The addition of lotion to the topsheet of absorbent articles is also known to provide benefits such as easier feces and menses clean up on the skin. For instance, U.S. Pat. No. 5,968,025 to Roe et al., WO 97/05908, WO 97/05909 and US 2006/140924 describe absorbent articles having lotioned topsheets for reducing adherence of feces to the skin, wherein the lotion compositions are primarily hydrophobic. U.S. Pat. No. 3,489,148 to Duncan et al. teaches a diaper comprising a hydrophobic and oleophobic topsheet wherein a portion of the topsheet is coated with a discontinuous film of oleaginous material. A disadvantage of the diapers disclosed in the Duncan et al. reference and other diapers treated with hydrophobic lotions is that the hydrophobic and oleophobic topsheets are slow in promoting transfer of urine to the underlying absorbent cores. Hydrophobic lotion compositions tend also to leave an undesirable greasy or slippery feel on the skin. Hydrophilic lotion compositions overcome many of these drawbacks. WO 05/035013, WO 00/64500, WO 00/64501, U.S. Patent Application 2002/120241 and U.S. Pat. No. 6,756,520 describe absorbent articles with hydrophilic lotion compositions for various uses, such as improving moisturization or lubrication, for reducing abrasion of skin, for improving skin health, for enhancing the barrier function of the skin and for prevention and alleviation of skin irritations. EP1992366A1 and EP1992367A1 describe absorbent articles with a hydrophilic lotion that transfers to the skin and limits the adherence of feces or menses to the skin (anti-stick properties). However, some of those hydrophilic lotion compositions that limit the adherence of feces or menses to the skin may incur process problems, in particular in high speed processes. Some of these hydrophilic lotions are not always effectively delivered to the skin, thus reducing their efficiency. When processing the hydrophilic compositions of the prior art in liquid state, at elevated temperature process conditions, these compositions may remain in liquid state after deposition onto a substrate for a substantial length of time, thus, affecting negatively the manufacturing process. In addition to the process issues, the longer the lotion composition stays in a liquid state, the more it tends to migrate into the article, so that a lesser amount is available for transfer to the skin or remains located between the skin and the skin-contacting surface of the absorbent article in use. Process issues associated with some hydrophilic lotion compositions are known. U.S. Pat. No. 6,515,029 teaches the use of process aids to decrease the time needed by the lotion compositions to solidify after deposition on the absorbent article. However, the offered solutions are not satisfactory for the hydrophilic lotions compositions that are intended for the reduction of adherence of feces and menses to the skin of the wearer; the offered solutions of the prior art can reduce the performance of these specific hydrophilic lotion compositions.

Thus, there is still an unmet need to provide absorbent articles to be applied to the skin or to be worn by a wearer, such as wipes, diapers, sanitary napkins and the like, that comprise a substrate, e.g., on the body facing surface of the absorbent article, comprising a hydrophilic lotion that is easily processable, even at high speed, and efficiently transfers to the skin to effectively reduce the adherence of feces or menses to it.

There remains a need for a substrate, comprised by, or forming, or used for manufacturing of, an absorbent article or a wipe, comprising a transferable lotion composition that is readily processable, even at high speed, whilst effectively reducing the adherence of menses or feces to the skin.

### SUMMARY OF THE INVENTION

A substrate comprises a lotion composition. The lotion composition may comprise from about 20 to about 80 weight percent of one or more compounds A which are liquid at 25° C. The compound(s) A may be selected from the group consisting of liquid polyethylene glycol, liquid polyethylene glycol derivatives, liquid polypropylene glycol, liquid polypropylene glycol derivatives; liquid polyhydric alcohol, liquid fatty acid esters comprising at least one fatty acid unit and at least one ethylene glycol unit, liquid fatty acid esters comprising at least one fatty acid unit and at least one propylene glycol unit, and mixtures thereof. The lotion composition may comprise from about 5 to about 50 weight percent of one or more compounds B which are solid at 25° C. The compound(s) B may be selected from the group consisting of solid polyethylene glycol derivatives, solid polypropylene glycol derivatives, solid alkoxylated non-ionic surfactants, solid glycerol esters, solid sorbitan and derivatives, solid sucrose esters and their derivatives, solid glucose esters and their derivatives, and mixtures thereof. The lotion composition may comprise from about 1 to about 40 weight percent of one or more crystallization accelerators C selected from the group consisting of C₁₄-C₂₂ fatty alcohols, C₁₂-C₂₂ fatty acids, solid fatty soap, waxes selected from the group consisting of carnauba, ozokerite, beeswax, candelilla, paraffin, ceresin, esparto, rezowax, isoparaffin, and mixtures thereof. The weight ratio of compound(s) A to crystallization accelerator(s) C may be from 3:2 to 10:1.

A process for manufacturing an absorbent article comprising a lotion composition limiting the adherence of feces or menses to the skin may comprise applying an effective amount of a lotion composition as described above to the body facing surface of a substrate forming the topsheet of the absorbent article. The lotion composition may be applied as a melt thereof. The lotion composition may be applied via spraying, printing, coating, extrusion or a combination thereof. The lotion composition may be resolidified. The resolidified lotion composition may form a solidified coating on the body facing surface of the substrate.

A method for reducing the adherence of feces or menses to the human skin may comprise providing a lotion composition. The lotion composition may comprise from about 20 to about 80 weight percent of one or more compounds A which are liquid at 25° C. The compound(s) A may be selected from the group consisting of liquid polyethylene glycol, liquid polyethylene glycol derivatives, liquid polypropylene glycol, liquid polypropylene glycol derivatives, liquid polyhydric alcohol, liquid fatty acid esters comprising at least one fatty acid unit and at least one ethylene glycol unit, liquid fatty acid esters comprising at least one fatty acid unit and at least one propylene glycol unit, and mixtures thereof. The lotion composition may comprise from about 5 to about 50 weight percent of one or more compounds B which are solid at 25° C. The compound(s) B may be selected from the group consisting of solid polyethylene glycol derivatives, solid polypropylene glycol derivatives, solid alkoxylated non-ionic surfactants, solid glycerol esters, solid sorbitan and derivatives, solid sucrose esters and their derivatives, solid glucose esters and their derivatives, and mixtures thereof. The lotion composition may comprise from about 1 to about 40 weight percent of one or more crystallization accelerators C. The crystallization accelerator(s) C may be selected from the group consisting of C₁₄-C₂₂ fatty alcohols, C₁₂-C₂₂ fatty acids, solid fatty soap, waxes selected from the group consisting of carnauba, ozokerite, beeswax, candelilla, paraffin, ceresin, esparto, rezowax, isoparaffin, and mixtures thereof. The weight ratio of compound(s) A to crystallization accelerator(s) C may be from 3:2 to 10:1. The method may comprise applying an effective amount of the lotion composition to the body facing surface of a substrate. The substrate may be used in the manufacture of, or as, or in an absorbent article. The absorbent article may be an article to be worn by a wearer. The method may comprise contacting the skin of the wearer with the absorbent article.

A method for assessing the suitability of a lotion composition in a high speed manufacturing process for making an absorbent article may comprise determining the crystallization onset temperature of the lotion composition using the Differential Scanning calorimetry method as described herein.

### BRIEF DESCRIPTION OF THE DRAWINGS

FIG. 1 is an illustrative example of a heat flow curve of a lotion composition in W/g versus temperature in ° C. (cooling data curve).

FIG. 2 is an illustrative example of the overlaying of a heat flow curve of a lotion composition in W/g versus temperature in ° C. (cooling data curve) with the curve of the first derivative of the heat flow with respect to the temperature in W/g.° C., used to determine the onset temperature of crystallization of a lotion composition.

### DETAILED DESCRIPTION OF THE INVENTION

As used herein “absorbent article” refers to devices which are intended to be placed against the skin of a wearer to absorb and contain the various exudates discharged from the body. Examples of absorbent articles include incontinence articles such as infant or adult diapers; pant-like diapers such as training pants; diaper holders; incontinence pads. Further examples of absorbent articles are feminine hygiene products such as sanitary napkins and panty-liners. In some embodiments, the absorbent articles are diapers, pant-like diapers, sanitary napkins or panty-liners.

As used herein “pad-type applicator” refers to devices which are intended to apply a lotion composition to a surface, such as the skin. For instance, the pad-type applicator may have an applicator pad portion having a surface coated with the lotion composition to be dispensed, and an upstanding finger grip portion.

### Substrate

The term “substrate” as used herein generally refers to a material comprised by, or forming, or used for manufacturing of the topsheet and/or the legs cuffs and/or the barrier cuffs and/or the side flaps and/or the side panels and/or the wings of an absorbent article. The substrate may also be a material suitable for manufacturing of, or suitable as, or in a wipe.

Suitable materials include woven and nonwoven materials (typically web materials, including woven or nonwoven webs of fibers); polymeric web materials such as (apertured formed) thermoplastic films, (apertured) plastic films, hydroformed thermoplastic films, porous foams, reticulated foams, reticulated thermoplastic films and thermoplastic scrims; paper tissue or combinations thereof.

Other substrates may include materials suitable for constituting the pad portion of a pad-like applicator, e.g., flexible, resilient polymeric foam material non-reactive with the lotion composition to be dispensed by the applicator. Exemplary resilient material include urethane foam, polyethylene foam and the like.

“Nonwoven material” as used herein refers to a manufactured web of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled.

Exemplary substrates may be or may comprise a nonwoven web material, whereby said nonwoven web may be manufactured by a wide number of known techniques. Non-limiting examples of techniques include spunbonding, carding, wet-laid, air-laid, melt-blown, needle-punching, mechanical entangling, thermo-mechanical entangling, hydroentangling, calender bonding, and combinations thereof.

Suitable substrates include web material (e.g., woven or nonwoven web) comprising natural fibers or synthetic fibers or combinations thereof. Examples of natural fibers may include cellulosic natural fibers, such as fibers from hardwood sources, softwood sources, or other non-wood plants. The natural fibers may comprise cellulose, starch and combinations thereof. The synthetic fibers can be any material, such as, but not limited to, those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof. Further, the synthetic fibers can be a single component (i.e., single synthetic material or mixture makes up entire fiber), bi-component (i.e., the fiber is divided into regions, the regions including two or more different synthetic materials or mixtures thereof and may include co-extruded fibers and core and sheath fibers) and combinations thereof. Bi-component fibers can be used as a component fiber of the web material, and/or they may be present to act as a binder for the other fibers present in the web material. Any or all of the synthetic fibers may be treated before, during, or after manufacture to change any desired properties of the fibers.

The substrate may be or may comprise a laminate web of two or more nonwoven webs. The laminate web may comprise spunbond layer(s) (S), and/or meltblown layer(s) (M), and/or carded layer(s) (C). Suitable laminate webs include, but are not limited to, SS, SSS, SMS or SMMS. The substrate may have a basis weight between about 5 to 100 g/m². Where the substrate is comprised by, or forms, or is used for manufacturing of the topsheet, the leg cuffs and/or the barrier cuffs and/or the side flaps and/or the wings of an absorbent article, the substrate may have, for example, a basis weight between about 5 to 100 g/m², or between about 10 to 40 g/m², or between about 10 to 30 g/m². Where the substrate is used as, or in a wipe, it may for example have a basis weight between about 15 to 100 g/m², or between about 30 to 95 g/m², or between about 40 to 85 g/m², or between about 45 to 75 g/m².

The substrate may be comprised by, or may form, or may be used for manufacturing of, the topsheet of an absorbent article. The topsheet is oriented towards and contacts the body of the wearer permitting bodily discharges to rapidly penetrate through it without allowing fluid to flow back through the topsheet to the skin of the wearer. The topsheet may be made of a hydrophobic material to isolate the wearer's skin from liquids which have passed through the topsheet and are contained in the absorbent core (i.e., to prevent rewet).

The substrate comprised by, or forming, or used for manufacturing of, the topsheet of an absorbent article may alternatively, or additionally, be apertured, i.e., the topsheet may have a plurality of apertures having an aperture size of at least about 0.2 mm². The topsheet may have an open area of at least about 10%, the open area being the sum of all apertures. The open area are may be determined by the procedure disclosed in WO 95/05139.

The substrate may be comprised by, or may form, or may be used for manufacturing of a topsheet that has one or more openings. Typically, the openings are large enough to let feces or menses pass to a void space underneath said topsheet, also referred to as anal cuff or vaginal cuff. For example, U.S. Patent Application No. 2006/0058766 A, filed on Sep. 13, 2005 discloses an absorbent article wherein the topsheet is provided with at least one opening adapted to receive fecal material. Such topsheets may be made of or may comprise a liquid impervious material, and thus, the substrate may be a liquid impervious material.

### Lotion Compositions

Lotion compositions may the adherence of feces or menses to the human skin. In one embodiment, they may improve the ease of removal of feces or menses after an absorbent article comprising said lotion composition has been used and removed from the wearer. Without being bound by theory, it is believed that the lotion composition may reduce the adhesive force between the soils or exudates and the skin surface because the adhesive forces may be smaller than the cohesive forces within the soils or exudates, thereby allowing the soils or exudates to detach from the skin surface upon application of a shear force (e.g., such as that generated by wiping).

When applied to a substrate comprised by, or forming, or used for manufacturing of, an absorbent article, the lotion compositions may be transferable to the wearer's skin by normal contact, wearer motion (thus creating friction), and/or body heat.

When applied to a substrate used in, or as a wipe, or when applied to a substrate comprised by the pad portion of a pad-like applicator, the lotion compositions are readily transferable from the substrate to the skin by applying a relatively low force to the substrate (e.g., wiping a surface such as the skin in the perianal area with the wipe or rubbing the skin in the perianal area with the product applying surface, i.e., pad portion, of a pad-type applicator).

The transfer or migration of the lotion compositions onto the skin for administration and/or deposition of the lotion compositions may result in a safe and effective amount of the lotion compositions being applied. The safe and effective amount of the lotion composition that will transfer or migrate to the body may depend on factors such as the type of lotion composition that is applied, the portion of the body facing surface of the substrate where the lotion composition is applied, and the type of absorbent article used to administer the lotion composition. In one embodiment, an effective amount is an amount which effects a reduction of the adherence of feces or menses to the human skin of a wearer wearing an absorbent article compared to the absorbent article without the lotion composition. An effective amount may be from about 0.0015 mg/cm² (0.01 mg/in²) to about 100.5 mg/cm² (100 mg/in²), from about 0.003 mg/cm² (0.02 mg/in²) to about 12.4 mg/cm² (80 mg/in²), or from about 0.02 mg/cm² (0.15 mg/in²) to about 7.75 mg/cm² (50 mg/in²), of the lotion composition to the absorbent article. Typically, a safe and effective amount of the lotion compositions is applied to an absorbent article such that at least about 0.00015 mg/cm² (0.001 mg/in²) to about 15.5 mg/cm² (100 mg/in²), from about 0.0006 mg/cm² (0.004 mg/in²) to about 11 mg/cm² (72 mg/in²), or from about 0.005 mg/cm² (0.03 mg/in²) to about 6.2 mg/cm² (40 mg/in²), of the lotion composition is transferred to the body during a single use of an absorbent article, which is typically about a three hour period. Any suitable method can be used in determining the amount of a lotion composition that is transferred to the body of a wearer during use of an absorbent article containing the lotion composition. Examples of methods for the calculation of transfer amounts of lotion compositions include Gas Chromatographic and other quantitative analytical procedures that involve the analysis of in vivo skin analog materials. A suitable Gas Chromatographic procedure is more fully described in WO 99/45973, Donald C. Roe et al, published Sep. 16, 1999.

The lotion compositions may be flowable (e.g., liquid) at suitable process conditions, e.g., above 50° C., or above 60° C., or above 80° C., or optionally above 100° C., but are typically non-fluid, i.e., solid, at 25° C., i.e., at ambient temperatures. The lotion compositions may have a final melting point (100% liquid) above potential “stressful” storage conditions that can be 45° C. or greater.

As used herein, non-fluid, i.e., solid, means that 1 g of the material, which is placed in the middle of a round glass plate having a diameter of 15 cm, does not run off a glass plate within 1 minute, when the glass plate is tilted at 45°, under conditions of 25° C. and 50% relative humidity. The consistency of the lotion compositions can be measured according to ASTM D5 test method which involves the use of a penetrometer to measure consistency.

The lotion compositions may be essentially non-aqueous. Non aqueous means that the lotion compositions either contain no water or they contain water only in minor amounts, such as less than 5 wt. %, or even less than 1 wt. %. However, these amounts refer to the lotion composition at the time when the absorbent article is produced, i.e., to the time the lotion composition is applied onto the absorbent article. The lotion compositions may be rather hygroscopic, and thus may be able to take up a significant amount of water from the surrounding atmosphere, particularly in an environment with high relative humidity. Thus, when the absorbent article has been stored for a relatively long time, such as several months or even years, it is possible that the amount of water contained in the lotion composition has increased to be more than 5 wt %.

A lotion composition may comprise:

a) from about 20 to about 80, or from about 30 to about 70, or from about 40 to about 60, weight percent of one or more compounds which are liquid at 25° C. (referred herein as compound A), selected from the group consisting of liquid polyethylene glycol, liquid polyethylene glycol derivatives, liquid polypropylene glycol, liquid polypropylene glycol derivatives, liquid polyhydric alcohol, liquid fatty acid esters comprising at least one fatty acid unit and at least one ethylene glycol unit, liquid fatty acid esters comprising at least one fatty acid unit and at least one propylene glycol unit, and mixtures thereof;

b) from about 5 to about 50, or from about 10 to about 30, or from about 15 to about 25, weight percent of one or more compounds which are solid at 25° C. (referred herein as compound B), selected from the group consisting of solid polyethylene glycol derivatives, solid polypropylene glycol derivatives, solid alkoxylated non-ionic surfactants, solid glycerol esters, solid sorbitan and derivatives, solid sucrose esters and their derivatives, solid glucose esters and their derivatives, and mixtures thereof;

c) from about 1 to about 40, or from about 3 to about 25, or from about 5 to about 20, weight percent of one or more crystallization accelerators (referred herein as compound C) selected from the group consisting of C₁₄-C₂₂ fatty alcohols, C₁₂-C₂₂ fatty acids, solid fatty soap, waxes selected from the group consisting of carnauba, ozokerite, beeswax, candelilla, paraffin, ceresin, esparto, rezowax, isoparaffin, and mixtures thereof;

and wherein the weight ratio of compound(s) A to crystallization accelerator(s) C is from 3:2 to 10:1.

In some embodiments, the lotion composition may comprise from about 20 to about 80, or from about 30 to about 70, or from about 40 to about 60, weight percent of one or more compounds A selected from the group consisting of liquid polyethylene glycol, liquid polyethylene glycol derivatives, liquid polypropylene glycol, liquid polypropylene glycol derivatives, liquid polyhydric alcohol and mixtures thereof, in combination with one or more compounds B and one or more crystallizations accelerators C as disclosed above.

In some embodiments, the lotion composition may comprise one or more compounds B selected from the group consisting of solid polyethylene glycol derivatives, solid polyethylene glycol fatty alcohol ethers and mixtures thereof, in combination with one or more compounds A and one or more crystallization accelerators C as disclosed above.

In some embodiments, the lotion composition may comprise:

a) from about 20 to about 80, or from about 30 to about 70, or from about 40 to about 60, weight percent of one or more compounds A selected from the group consisting of liquid polyethylene glycol, liquid polyethylene glycol derivatives, liquid polypropylene glycol, liquid polypropylene glycol derivatives, liquid polyhydric alcohol and mixtures thereof;

b) from about 5 to about 50, or from about 10 to about 30, or from about 15 to about 25, weight percent of one or more compounds B selected from the group consisting of solid polyethylene glycol derivatives, solid polyethylene glycol fatty alcohol ethers and mixtures thereof;

c) from about 1 to about 40, or from about 3 to about 25, or from about 5 to about 20, weight percent of one or more crystallization accelerators C selected from the group consisting of C₁₄-C₂₂ fatty alcohols, C₁₂-C₂₂ fatty acids and mixtures thereof; and wherein the weight ratio of compound(s) A to crystallization accelerator(s) C is from 3:2 to 10:1.

In some embodiments, the lotion composition may comprise:

a) from about 20 to about 80, or from about 30 to about 70, or from about 40 to about 60, weight percent of a polyethylene glycol compound as compound A;

b) from about 5 to about 50, or from about 10 to about 30, or from about 15 to about 25, weight percent of a polyethylene glycol fatty alcohol ether as compound B;

c) from about 1 to about 40, or from about 3 to about 25, or from about 5 to about 20, weight percent of a C₁₄-C₂₂ fatty alcohol compound as a crystallization accelerator; and wherein the weight ratio of the polyethylene glycol compound to crystallization accelerator is from 3:2 to 10:1.

Compound(s) A, B, and C are further described below.

### Liquid Compound(s) at 25° C. (Compound A)

The lotion composition may comprise one or more compounds A which are liquid at 25° C. as described below. In some embodiments, the lotion composition may comprise one compound A which is liquid at 25° C. In some embodiments, the lotion composition may comprise a mixture of compounds A which are liquid at 25° C.

The amount of said liquid compound A, or of said mixture of compounds A, is comprised between about 20 to about 80 weight percent, or between about 30 to 70 weight percent, or between about 40 to 60 weight percent of the total weight lotion composition.

The liquid compound(s) A may be selected from the group consisting of liquid polyethylene glycol, liquid polyethylene glycol derivatives, liquid polypropylene glycol, liquid polypropylene glycol derivatives, liquid polyhydric alcohol, liquid fatty acid esters comprising at least one fatty acid unit and at least one ethylene glycol unit, liquid fatty acid esters comprising at least one fatty acid unit and at least one polypropylene unit, and mixtures thereof.

### Liquid Polyethylene Glycols

Liquid polyethylene glycols are liquid at 25° C. They are made from at least 3 units of ethylene glycol and have the general formula HO—(CH2-CH2-O)ₓ—H with x being a number of from 3 to 15 or from 8 to 12. The molecular weight, expressed in Dalton, (weight average) is from 100 to less than 720, or from 350 to 700. Some liquid polyethylene glycols are known as PEG-4, PEG-6, PEG-7, PEG-8, PEG-9, PEG-10, PEG-12 and PEG-14. Suitable trade products are, for example, Polyglykol 400 of Clariant with an average molecular weight of 380 to 410 or Polyglykol 600 with an average molecular weight of 570 to 630 Daltons.

### Liquid Polyethylene Glycol Derivatives

The liquid polyethylene glycol derivatives are liquid at 25° C. Liquid polyethylene glycol derivatives are mono- or di-ester or ether end-capped polyethylene glycols. The end-capping group(s) may be a methyl, an ethyl and/or a propyl group. Suitable liquid polyethylene glycol derivatives include polyethylene glycol monomethyl ether such as available as Polygkycol M400 from Clariant or polyethylene glycol dimethyl ether with a molecular weight of 500 Daltons as available from Sigma-Aldrich.

### Liquid Polypropylene Glycol

Liquid polypropylene glycols are liquid at 25° C. They are made from at least 3 units of propylene glycol. Some liquid polypropylene glycols are known as PPG-9, PPG-17, PPG-20, PEG-26 and PPG-30.

### Liquid Polypropylene Glycol Derivatives

The liquid polypropylene glycol derivatives are liquid at 25° C. The liquid polypropylene glycol derivatives are mono- or di-ester or ether end-capped polypropylene glycols. The end-capping group(s) may be a methyl, an ethyl, a propyl and/or a butyl group. Suitable liquid polypropylene glycol derivatives include PPG-2 butyl ether as available from Dow Chemical under the trade name Dowanol DPnB.

### Liquid Polyhydric Alcohol

Liquid polyhydric alcohol are organic compounds having at least 2 carbon atoms and at least two alcoholic hydroxy groups and which are liquid at 25° C. Examples are glycerol, ethylene glycol, diethylene glycol, propylene glycol, butylene glycol, dipropylene glycol, methyl propanediol, and the like.

### Liquid Fatty Acid Esters Comprising at Least One Fatty Acid Unit and at Least One Ethylene Glycol Unit

Suitable liquid ethylene glycol fatty acid esters are, for example, the esters of one or more ethylene glycol units and one or two fatty acids. They have the general formula R¹—(OCH2CH2)ₘO—R² where R¹ and R² are hydrogen or fatty acid residues with, e.g., from 6 to 30, or from 8 to 22, carbon atoms and can be the same or different with the proviso that not both are hydrogen; and m is a number of at least 1. In some embodiments, R¹ and R² are different and m is 1, 2, or 3. Some ethylene glycol esters are known, for example, as diethylene glycol diethylhexanoate/diisononanoate, diethylene glycol diisononanoate, diethylene glycol dilaurate, diethylene glycol dioctanoate/diisononanoate and diethylene glycol distearate. Suitable trade product mixtures containing ethylene glycol esters are for example DERMOL MO or DERMOL 489.

### Liquid Fatty Acid Esters Comprising at Least One Fatty Acid Unit and at Least One Propylene Glycol Unit

Suitable liquid propylene glycol fatty acid esters are for example the esters of one or more propylene glycol units and one or two fatty acids.

### Solid Compound(s) at 25° C. (Compound B)

The lotion composition may comprise one or more compounds B which are solid at 25° C. as described below. Solid compound as used herein means that the compound has an average melting point of at least about 30° C. or at least about 35° C. or at least about 40° C. In some embodiments, the lotion composition may comprise one solid compound B. In some embodiments, the lotion composition may comprise a mixture of compounds B.

The amount of said compound B, or of said mixture of compounds B may be between about 5 to about 50 weight percent, or between about 10 to 40 weight percent, or between about 15 to 30 weight percent of the total weight lotion composition.

The solid compound(s) B may be selected from the group consisting of solid polyethylene glycol derivatives, solid polypropylene glycol derivatives, solid alkoxylated non-ionic surfactants, solid glycerol esters, solid sorbitan and derivatives, solid sucrose esters and their derivatives, solid glucose esters and their derivatives, and mixtures thereof.

The solid compound B may have a Hydrophile-Lipophile Balance value of at least 10.

### Solid Polyethylene Glycol Derivatives

Solid polyethylene glycol derivatives are made from at least 16 units of ethylene glycol, e.g., from 16 to 220 units of ethylene glycol. The solid polyethylene glycol derivatives are mono- or di-ester or ether end-capped polyethylene glycols. The end-capping group(s) may be a methyl, an ethyl and/or a propyl group.

The molecular weight expressed in Dalton (weight average) may be above 720, or from 720 to 100000. Typical solid polyethylene glycols derivatives are known as Brij 700, available from Croda, and Myrj 59 from Croda.

### Solid Polypropylene Glycol Derivatives

Solid polypropylene glycol derivatives are made from units of propylene glycol. The solid polypropylene glycol derivatives are mono- or di-ester or ether end-capped polyethylene glycols. The end-capping group(s) may be a methyl, an ethyl and/or a propyl group.

### Alkoxylated Solid Nonionic Surfactants

Suitable alkoxylated solid nonionic surfactants include, for example, solid polyalkylene glycol fatty alcohol ethers, such as solid polyethylene glycol fatty alcohol ethers. Polyethylene glycol fatty alcohol ethers have the general formula R(O—CH2-CH2)ₘOH, where R represents an alkyl group or a mixture of alkyl groups with for example 8 to 30 or 12 to 22 carbon atoms. The average degree of ethoxylation may be from 2 to 200, or at least 10, at least 20, or at least 30. The polyethylene glycol fatty alcohol ethers may be nonionic surfactants with Hydrophile-Lypophile Balance values of at least 10, or at least 12, e.g., 13 to 17. Suitable trade products are for example BRIJ 76, BRIJ 78, and BRIJ 700. Other suitable alkoxylated solid nonionic surfactants include, e.g., ethoxylated alcohols, ethoxylated fatty acids and ethoxylated fatty esters.

### Crystallization Accelerator (Compound C)

The lotion composition may comprise one or more crystallization accelerators C as described below. In some embodiments, the lotion composition comprises one crystallization accelerator C. In some embodiments, the lotion composition comprises a mixture of crystallization accelerators C.

The amount of said crystallization accelerator C, or of said mixture of crystallization accelerator C may be between about 1 to about 40 weight percent, or between about 3 to 30 weight percent, or between about 5 to 20 weight percent of the total weight lotion composition.

The crystallization accelerator(s) C may be selected from the group consisting of C₁₄-C₂₂ fatty alcohol, C₁₂-C₂₂ fatty acid, solid fatty soap, waxes selected from the group consisting of carnauba, ozokerite, beeswax, candelilla, paraffin, ceresin, esparto, rezowax, isoparaffin, and mixtures thereof. In some embodiments, the crystallization accelerator(s) C may be selected from the group consisting of C₁₄-C₂₂ fatty alcohol, C₁₂-C₂₂ fatty acid and mixtures thereof.

### Fatty Compounds

The fatty compound may be selected from the group consisting of solid fatty alcohol, solid fatty acid and/or solid fatty soap. The fatty compound is solid at 25° C.

In one embodiment, the fatty compound is selected from the group consisting of C₁₄-C₂₂ fatty alcohol, C₁₂-C₂₂ fatty acid, solid fatty soap and mixtures thereof.

Exemplary fatty compounds include saturated, linear fatty compounds. Examples of solid fatty acids are decanoic acid, lauric acid, myristic acid, palmitic acid, stearic acid, arachidic acid or behenic acid.

Exemplary solid fatty alcohols are linear, unsaturated 1-alkanols with at least 14 carbon atoms. Examples of solid fatty alcohols are myristyl alcohol, cetyl alcohol, stearyl alcohol, arachidyl alcohol or behenyl alcohol.

The solid fatty soaps are metallic soaps which are metal salts of fatty acids. The fatty acid components of the fatty soaps are the same as mentioned above. Suitable metal cations are sodium, potassium, lithium, aluminium, magnesium, calcium, manganese, iron, zirconium, cerium, zinc, cobalt or vanadium. Some metallic soaps may have low water solubility, such as the calcium or magnesium salts, e.g., calcium stearate.

### Waxes

The waxes may be selected from the group consisting of carnauba, ozokerite, beeswax, candelilla, paraffin, ceresin, esparto, rezowax, isoparaffin and other known mined and mineral waxes.

Surprisingly, it has been found that the lotion compositions disclosed herein provide the right performance in terms of processability and anti-stick. Indeed, unexpectedly, it has been found that the specific combinations of one or more compounds A as disclosed above with one or more compounds B as disclosed above, and with one or more crystallization accelerators C as disclosed above, provided that the weight ratio compound(s) A to crystallization accelerator(s) C be between 3:2 and 10:1, provide lotion compositions which may be easily processable, even at high speed (e.g., above 400 absorbent articles per minute), and which may reduce effectively the adherence of feces or menses to the skin (anti-stick performance). Lotion compositions not fulfilling those conditions were found to not exhibit the desirable anti-stick performances and/or to not solidify sufficiently quickly to be considered suitable for use in absorbent articles manufacturing processes.

Lotion compositions are considered easily processable at high speed where they quickly solidify after their deposition in a melted state on a substrate thus, enabling a rapid handing of the substrate in the subsequent steps of the manufacturing processes of the absorbent articles or wipes. The determination of the crystallization onset temperature of a lotion composition according to the Differential Scanning Calorimetry Method, disclosed in the test method section, has been found to ideally picture the behavior of a lotion composition in practice, i.e., how quick it crystallizes once applied on a substrate and thus, to provide an excellent indication of the suitability of a lotion composition for use in a high speed process. The lotion compositions disclosed herein exhibit crystallization onset temperatures that are representative of lotions compositions easily processable at high speed.

As mentioned above, the lotion compositions disclosed herein exhibit desirable anti-stick performance. Typically, the more efficient the lotion composition is, the lower the percentage of residual ABM on a transparency film is. An average residual ABM of less than 10%, as measured by the method described in the Test Method section, is illustrative of particularly good anti-stick performance.

Because of their anti-stick properties, the lotion compositions disclosed herein may be suitably used in methods for reducing the adherence of feces or menses to the human skin. Thus, the adherence of fences or menses to the human skin may be reduced by providing a lotion composition as disclosed above, applying an effective amount of the lotion composition to the body facing surface of a substrate used in the manufacture of, or as, or in an absorbent article to be worn by a wearer, and contacting the skin of the wearer with the absorbent article. By normal contact, wearer motion, and/or body heat, the lotion composition transfers to the skin and limits the adherence of feces or menses.

Additionally, as a further benefit, it has also been found that the lotion composition disclosed herein exhibits desired substrate wetting performance on hydrophobic and hydrophilic substrates. Indeed, the lotion compositions desirably wet/spread on hydrophobic or hydrophilic substrates upon application and are, therefore, less likely to fall or rub off of the substrate during further processing.

Furthermore, the lotion compositions disclosed herein provide desirable wetting properties to the substrate due to their suitable hydrophilic character. Insuring the wetting properties of the substrate is important, notably when the substrate is used in an absorbent article, such as a diaper: the lotion compositions deposited on a substrate used as, or in a topsheet, may be sufficiently wettable to ensure that liquids (e.g., urine) will transfer through the topsheet rapidly. This decreases the likelihood that body exudates will flow off the lotion coating rather than being absorbed by the absorbent core.

### Optional Ingredients

### Particulate Material

In some embodiments, the lotion composition comprises at least one particulate material for further reducing the adherence of feces or menses to the skin. The term “particulate material” as used herein refers to a component of the lotion composition that is insoluble, non-molecularly dispersible in the lotion composition at room temperature and at process conditions, e.g., above 50° C., or above 60° C., or above 80° C., or above 100° C. Thus, the particulate material has a melting temperature above the processing temperature of the lotion composition, hence it remains particulate during application onto the substrate as disclosed above. The particulate material may be such that it remains particulate when in contact with the skin and/or when in contact with urine, menses or feces. Hence, the particulate material may be water-insoluble.

The particulate material may have any mean particle size between 1 nanometer to 2 mm, between 1 nanometer to 500 micrometers, between 0.1 micrometer to 2 mm, between 50 nanometers to 1 micrometer, or any range or individual value within any of the ranges set forth herein. The minimum mean particle size may be at least 0.1 micrometer or at least 1 micrometer, or at least 10 micrometers, or at least 20 micrometers, and up to about 500 micrometers, or in some embodiments, up to about 100 micrometers, and in other embodiments, up to about 30 micrometers. In some embodiments, the lotion composition to be applied and/or the applied coating may comprise particles whereof less than 25% of the particles have an equivalent diameter of greater than 100 microns. In some embodiments, the lotion composition to be applied and/or the applied coating may comprise particles whereof less than 25% of the particles have an equivalent diameter of less than 5 microns. In yet another embodiment, the lotion composition to be applied and/or the applied coating may comprise particles whereof less than 25% of the particles have an equivalent diameter of less than 100 microns.

The particulate material may be present in the lotion composition at a level from 0.05% to 25% (by weight of the lotion composition), from 0.05% to 15%, from 0.05% to 5%, from 0.1% to 25%, or from 0.25% to 20%, but typically from 0.5% to 10%, or even up to 5%, by weight.

Suitably, the particulate material may have a particle density between about 0.5 gram/cm³ and about 2.5 gram/cm³. The density may be between about 0.5 gram/cm³ and about 2.0 gram/cm³, or between 0.8 gram/cm³ and about 1.5 gram/cm³. In one embodiment, the density may be less than about 1 gram/cm³ so as to minimize particle settling, and the density may be greater than about 0.8 gram/cm³ so as to minimize particle floatation.

In one embodiment, the lotion composition may comprise inorganic particulate materials, including alumina silicates, silicates, silicas, mica and/or talc. Clays may also be used. However, the particulate material may be an organic material. The particulate material may be a non-active and/or non-reactive material. The particles of the particulate material may be porous, or non-porous. The particles may have any shape. They may have a smooth surface, and they may be spherical or plate-like particles. The particles may comprise a coating agent on their surface or part thereof, for example, a surfactant to change its properties, e.g., hydrophilicity. The particles, in particular when they are oleofinic, may include a melt-additive, which may be added during the manufacturing of the particles.

Suitable particulate materials include, but are not limited to, polystyrene particles, polypropylene and/or polyethylene (co)polymer particles, polytetrafluoroethylene particles, polymethylsilsesquioxane particles, and nylon particles. Suitable commercially available particulate materials include, but are not limited to, polyethylene particles, available from Honeywell International of Morristown, N.J. under the trade name ACUMIST; polymethyl methacrylate particles (microspheres), available from KOBO of South Plainfield, N.J. as BPA; lactone cross polymer particles (microspheres), available from KOBO as BPD; Nylon-12 particles (microspheres), available from KOBO as NYLON SP; polymethylsilsesquioxane particles (microspheres), available from KOBO as TOSPEARL; cellulose particles (microspheres), available from KOBO as CELLO-BEADS; polytetrafluoroethylene powders, available from Micro Powders, Inc. of Tarrytown, N.Y. as MICROSLIP; blends of natural wax and micronized polymers as are available form Micro Powders, as MICROCARE; and particles of a copolymer of vinylidene chloride, acrylonitrile and methylmethacrylate available as EXPANCEL from Expancel, Inc. of Duluth, Ga. Micronized waxes, such as are available from Micro Powders as MICROEASE, may also be incorporated. Exemplary polyolefin particles (powders) are available from Equistar Chemical Corp., Houston, Tex. as MICROTHENE, such as MICROTHENE FN510-00 from Equistar.

### Optional Ingredients Enhancing the Benefits to the Wearer

In order to better enhance the benefits to the wearer, additional ingredients can be included in the lotion compositions. For example, the classes of ingredients that may be used and their corresponding benefits include, without limitation: antifoaming agents (reduce the tendency of foaming during processing); antimicrobial actives; antifungal actives; antiseptic actives; antioxidants (product integrity); astringents—cosmetic (induce a tightening or tingling sensation on skin); astringent—drug (a drug product which checks oozing, discharge, or bleeding when applied to skin or mucous membrane and works by coagulating protein); biological additives (enhance the performance or consumer appeal of the product); colorants (impart color to the product); deodorants (reduce or eliminate unpleasant odor and protect against the formation of malodor on body surfaces); external analgesics (a topically applied drug that has a topical analgesic, anesthetic, or antipruritic effect by depressing cutaneous sensory receptors); fragrances (consumer appeal); humectants (increase the water content of the top layers of the skin); natural moisturizing agents (NMF) and other skin moisturizing ingredients known in the art; opacifiers (reduce the clarity or transparent appearance of the product); and skin conditioning agents.

### Absorbent Articles Comprising the Lotion Composition

The lotion compositions may be applied to a substrate comprised by, or forming, or used for manufacturing of, an absorbent article. The lotion composition may be applied to the body facing surface of the substrate.

### Diaper

In the following, a diaper is described as one embodiment of an absorbent article. However, as the skilled person is aware of, most of the components and materials described herein are also applicable to other incontinence products, such as training pants or adult incontinence products.

The diaper has a longitudinal axis and a transverse axis. The diaper has further an inner, body facing surface and an outer, garment facing surface opposed to the inner surface.

One end portion of the diaper may be configured as a front waist region (which is the front one-third of the article, having one third of the length of the article). The opposite end portion may be configured as a back waist region (back one-third) of the diaper, having one third of the length of the article. An intermediate portion of the diaper may be configured as a crotch region (center one-third), which extends longitudinally between the front and back waist regions, also having one third of the length of the article. The crotch region is that portion of the diaper which, when the diaper is worn, is generally positioned between the wearer's legs.

The chassis of the diaper comprises the main body of the diaper. The chassis may comprise a topsheet, which may be liquid pervious, and which may, comprise or be made of a substrate comprising a lotion composition as described herein. The chassis may comprise a backsheet. The chassis may further include an absorbent core encased between the topsheet and the backsheet. The backsheet may be a liquid impervious backsheet, as known in the art. In one embodiment, the liquid impervious backsheet comprises a thin plastic film, such as a thermoplastic film having a thickness of about 0.01 mm to about 0.05 mm. Suitable backsheet materials may comprise breathable material, which permits vapors to escape from the absorbent article while still preventing exudates from passing through the backsheet. Suitable backsheet films include those manufactured by Tredegar Industries Inc. of Terre Haute, Ind. and sold under the trade names X15306, X10962 and X10964. The backsheet, or any portion thereof, may be elastically extendable in one or more directions. The absorbent core may comprise any absorbent material that is generally compressible, conformable, non-irritating to the wearer's skin, and capable of absorbing and retaining liquids such as urine and other body exudates.

The absorbent core may be manufactured in a wide variety of sizes and shapes (e.g., rectangular, hourglass, “T”-shaped, asymmetric, etc.) and from a wide variety of liquid-absorbent materials commonly used in disposable diapers and other absorbent articles such as comminuted wood pulp, which may be referred to as airfelt. Examples of other suitable absorbent materials include creped cellulose wadding, meltblown polymers including coform, cross-linked cellulosic fibers, tissue including tissue wraps and tissue laminates, absorbent foams, absorbent sponges, superabsorbent polymers, absorbent gelling materials, or any equivalent material or combinations of materials. The configuration and construction of the absorbent core may also be varied, e.g., the absorbent core may have varying caliper zones, a hydrophilic gradient, a superabsorbent gradient, or lower average density and lower average basis weight acquisition zones; or may comprise one or more layers or structures. The total absorbent capacity of the absorbent core should, however, be compatible with the design loading and the intended use of the diaper. Further, the size and absorbent capacity of the absorbent core may be varied to accommodate wearers ranging from infants through adults.

The diaper may have leg cuffs and/or barrier cuffs, which may, comprise or be made of a substrate comprising a lotion composition as described herein. The diaper may have a pair of opposing (elasticated) leg cuffs, including so-called side panels, and/or a pair of opposing (elasticated) barrier cuffs that provide improved containment of liquids and other body exudates. The cuffs of a pair may be mirror images of one another in the y-axis (longitudinal axis) of the article. Suitable cuffs are described in for example U.S. Pat. No. 3,860,003; U.S. Pat. No. 4,808,178; U.S. Pat. No. 4,909,803; U.S. Pat. No. 4,695,278; and U.S. Pat. No. 4,795,454.

The diaper may comprise a front and back waist band and/or a fastening system, typically joined to the waistband, as known in the art. Fastening systems may comprise fastening tabs and landing zones, wherein the fastening tabs are attached or joined to the back region of the diaper and the landing zones are part of the front region of the diaper.

Processes for assembling the diaper include conventional techniques known in the art for constructing and configuring disposable absorbent articles. For example, the backsheet and/or the topsheet can be joined to the absorbent core or to each other by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn. under the designation HL-1258 or H-2031.

### Feminine Hygiene Product

In the following, a feminine hygiene product is described (e.g., sanitary napkin or panty-liner). A feminine hygiene product may comprise a topsheet which may comprise or be made of said substrate comprising the lotion composition a backsheet, and an absorbent core positioned between the topsheet and backsheet; each component having a body facing surface and a garment facing surface. The topsheet may be made of a substrate comprising the lotion composition as described herein. The backsheet can be any known or otherwise effective backsheet material, provided that the backsheet prevents external leakage of exudates absorbed and contained in the feminine hygiene article. Flexible materials suitable for use as the backsheet include, but are not limited to, woven and nonwoven materials, laminated tissue, polymeric films such as thermoplastic films of polyethylene and/or polypropylene, composite materials such as a film-coated nonwoven material, or combinations thereof, as is well known in the art of making feminine hygiene articles such as sanitary napkins, pantiliners, and the like.

The feminine hygiene product may comprise an absorbent core. The absorbent core may be positioned between the topsheet and the backsheet. The size and shape of the absorbent core can be altered to meet absorbent capacity requirements, and to provide comfort to the wearer/user. The absorbent core can be any liquid-absorbent material known in the art for use in absorbent articles, provided that the liquid-absorbent material can be configured or constructed to meet absorbent capacity requirements. Non-limiting examples of liquid-absorbent materials suitable for use as the absorbent core include comminuted wood pulp, which is generally referred to as airfelt; creped cellulose wadding; absorbent gelling materials including superabsorbent polymers such as hydrogel-forming polymeric gelling agents; chemically stiffened, modified, or cross-linked cellulose fibers; meltblown polymers including coform; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof, as is well known in the art of making feminine hygiene articles such as sanitary napkins, pantiliners, and the like.

The feminine hygiene product may comprise wings, which may enable attachment to the underwear of the wearer. The wings may be made of, or comprise, a substrate comprising the lotion composition disclosed herein. The sanitary napkins and/or panty-liners herein may comprise a fastening means comprised by the backsheet and/or by the wings. Adhesive attachment means may be present on or attached to at least the backsheet.

### Wipes Comprising the Lotion Composition

In the following a wipe is described. The wipe may be made of a nonwoven substrate comprising a lotion composition as described herein. For instance, the lotion composition may be contacted with a substrate such as Fibrella 3160, a 58 grams/m² nonwoven comprising a blend of 40% viscose fibers and 60% polypropylene fibers as is available from Suominen of Tampere, Finland.

Whilst not limited to a particular use, where the substrate is used for manufacturing of wipes (e.g., wet wipes), it may be intended for cleaning the body, in particular the peri-anal area after defecation and/or the external genital area after urination of babies, toddlers, and adults. Other examples of use of the substrate when in the form of wipes include feminine hygiene wipes.

This disclosure also encompasses the combination of absorbent articles such as those described herein (e.g., diaper including a topsheet comprising a substrate comprising the lotion composition disclosed herein) with wipes comprising the lotion composition disclosed herein.

This combination includes combined uses or sales. The absorbent articles comprising a substrate comprising the lotion composition as described herein may be packaged with one or more wipes. In one embodiment, one or more wipes may be packaged in a first package and one or more absorbent articles may be packaged in a second package. The first and second packages may be packaged together or they may be held in assembly by any means. In another embodiment, one absorbent article may be packaged with one or more wipes as one individual package, which is especially convenient for users en-route, where it might be desirable to carry only one absorbent article and one or more wipes. The absorbent articles and the wipes may also be co-marketed, e.g., designed and/or advertised to be sold and/or used together.

### Process for Applying the Lotion Composition on a Substrate

The lotion compositions disclosed herein can be applied to a substrate by any known or otherwise effective technique for distributing a lotion composition onto a substrate. The lotion composition may be applied from a melt thereof to a substrate comprised by, or forming, the topsheet and/or the leg cuffs and/or the barrier cuffs and/or the wings and/or the side flaps of an absorbent article, or to a substrate used as, or in a wipe. It may also be applied to the pad portion of a pad-like applicator.

Since the lotion composition melts at above-ambient temperatures, it may be applied as a heated lotion composition to the substrate. The lotion composition may be heated to a temperature in the range from about 40° C. to about 100° C., or from 50° C., or from 60° C., or even from to 90° C., to about 100° C., prior to being applied to the substrate. Then, once the lotion composition has been applied to the substrate comprised by, or forming, the topsheet and/or the leg cuffs and/or the barrier cuffs and/or the wings and/or the side flaps or used as, or in, a wipe, it may be allowed to cool and solidify rapidly (e.g., less than 1 s, or 2 s, or 5 s) to form solidified coating on the surface of the substrate. Resolidification of the lotion composition may occur almost instantaneously, without the need for external cooling means such as chill rolls. However, external means such as chill rolls, either before or after the application of the melt, can be used if desired to further accelerate resolidification. Non-limiting examples of methods of applying the lotion compositions to a substrate include spraying, printing (e.g., flexographic printing), coating (e.g., contact slot coating and gravure coating), extrusion, or combinations of these application techniques. The lotion composition can be applied directly to the substrate comprised by, or forming, or used for manufacturing of, the topsheet and/or leg cuff and/or barrier cuff and/or the wings and/or the side flaps of an absorbent article or it may be applied to another material or component which is then adhered to the desired portion of the absorbent article (such as a calender roll).

The lotion composition may be applied uniformly or non-uniformly to the body facing surface of the substrate. By body facing surface of a substrate as used herein, it is meant the outer surface or outer surfaces of a substrate that in use are in contact with the skin of the wearer. By non-uniform it is meant here the amount and/or pattern of distribution of the lotion composition can vary over the substrate; including, for example, an embodiment wherein one or more portions of the treated surface of the substrate herein can have a greater amount of the lotion composition than other one or more other portions (e.g., some portions comprise a higher basis weight of the lotion composition than other portions, such as, for example, at least 10%, or at least 20% higher), including portions of the surface that do not have lotion composition on them. Such portions may have any of the dimensions as, for example, defined below. As the lotion composition limits adherence for body exudates such as feces or menses, in one embodiment, it may be, for example, comprised in those portions of the substrate comprised by, or forming, or used for manufacturing of the topsheet or the cuffs, which lie adjacent the skin areas of the wearer, which typically are contaminated with feces and menses. Thus, the lotion composition could, for example, be comprised in those portions which lie adjacent the buttocks and/or the whole groove length of the wearer in use, preferably also in the region of the genitals. The amount of lotion composition applied can be the same for the rear third of the article (i.e., a third of the longitudinal extension of the absorbent articles starting from the outer edge of the chassis in the rear waist region), the central third of the article, and the front third of the article. Alternatively, the amount of lotion composition applied can be different for the rear, central and front third of the absorbent article.

Where the lotion composition is applied non-uniformly, it can be applied intermittently, i.e., discontinuously. Any pattern, including pattern of portions, as described herein above, comprising the lotion composition, may be utilized, including, for example, application of a pattern of (or a pattern of portion having the shape of) small droplets (obtained via, e.g., spraying), discrete figures of any shape or size, such as round, oval, rectangular, triangular, star-shaped, heart-shaped or shaped in the form of an animal (obtained via, e.g., gravure printing), alternating stripes that run in the longitudinal or lateral direction of the article, etc. Also, the substrate can comprise figures of different shapes and/or of different sizes. By alternating stripes, it is meant portions in which the lotion composition is applied as stripes separated by portions which have no lotion composition applied.

The portions, stripes and/or other discrete figures and/or droplets may have a width from between 0.1 mm to about 50 mm, from between 0.1 to about 30 mm, from between 0.5 mm to about 50 mm, from about 0.5 mm to about 40 mm, from between 2 mm to about 40 mm, from between 2 mm to about 20 mm, from between 2 mm to about 15 mm, or from between 5 mm to about 20 mm. The spacing between the stripes having no lotion composition applied may have a width from between 0.1 mm to about 100 mm, from about 0.1 mm to about 50 mm, from between 0.1 to about 30 mm, from between 0.5 mm to about 50 mm, from about 0.5 mm to about 40 mm, from between 2 mm to about 40 mm, from between 2 mm to about 20 mm, from between 2 mm to about 15 mm, or from between 5 mm to about 20 mm. The portions in the form of stripes and/or discrete figures and/or droplets may have a length of at least 0.5 mm, or of at least 2 mm, or of at least 5 mm. In one embodiment, if the portions comprising the lotion composition are in the form of stripes, they may extend from one edge of the substrate to the opposite edge. The stripes may extend into the rear waist region of the substrate to the extent that they also cover the buttocks and/or most of the groove length. Where the lotion composition is applied in the form of a pattern of figures, the density and/or the size of the figures and/or the basis weight of the lotion composition comprised by the figures may be higher, such as 10% higher, or 20% higher, in those portions lying against the areas (e.g., the central third and rear third of the absorbent article) typically affected with feces smeared against the skin.

The lotion compositions may be applied to a substrate to result in effective amounts being transferred onto the skin, i.e., to impart the desired lotion composition benefits. For example, the lotion composition may be applied to a substrate, e.g., to the body facing surface of a substrate used in the manufacture of, or as, or in an absorbent article such as the topsheet of an absorbent article, in an amount ranging from about 0.0015 mg/cm² (0.01 mg/in²) to about 100.5 mg/cm² (100 mg/in²), or from about 0.003 mg/cm² (0.02 mg/in²) to about 12.4 mg/cm² (80 mg/in²), or from about 0.02 mg/cm² (0.15 mg/in²) to about 7.75 mg/cm² (50 mg/in²).

### Examples

The following lotion compositions were prepared by mixing the following melted (i.e., liquid) components together (table 1).

Their anti-stick behavior was investigated according to the anti-stick test method as described below with the artificial bowel movement lotion as described below. The results are summarized in table 1. The more efficient the lotion composition is, the lower the percentage of residual ABM on a transparency film is. An average residual ABM of less than 10% is illustrative of particularly good anti-stick performance.

The crystallization onset temperature of the following lotion compositions was measured according to the Differential Scanning calorimetry method as described below. The lotion compositions were applied on a substrate made of polypropylene. The results are summarized in table 1.

TABLE 1
Lotion compositions and their average residual AMB (%) on a transparency film and
their crystallization onset temperature.

| | | Steareth- | Stearyl | Stearic | Average residual | | Onset of |
|---|---|---|---|---|---|---|---|
| Lotion | PEG-400¹ | 100² | alcohol³ | acid⁴ | ABM (%) | | crystallization |
| treatment | (%) | (%) | (%) | (%) | (sdt error) | | (° C.) |
| No | — | — | — | — | 35.1 | (3.3) | — |
| treatment | | | | | | | |
| Lotion 1 | 50 | 20 | 30 | — | 5.8 | (2.2) | 51.7 |
| Lotion 2 | 50 | 40 | 10 | — | 2.5 | (0.3) | 48.9 |
| Lotion 3 | 50 | 20 | — | 30 | 8.3 | (4.0) | 42.3 |
| Lotion 4 | 50 | 40 | — | 10 | 3.0 | (1.0) | 32.5 |
| Lotion 5* | 50 | 5 | 45 | — | 22.6 | (2.5) | 53 |
| Lotion 6* | 50 | 5 | | 45 | 19.9 | (3.1) | 45.3 |

*Lotion composition not according to the present disclosure
¹supplied by Sasol North America (Houston, TX);
²supplied by Croda Inc. (Edison, NJ);
³supplied by Procter & Gamble (Cincinnati, OH);
⁴supplied by Procter & Gamble (Cincinnati, OH).

### Test Methods

### Anti-Stick Method (Anti-Stick Performance)

This method may be used for assessing the adhesion of soils or exudates to a substrate by quantifying the percentage of residual artificial pasty bowel movement (“ABM”) left on a transparency film after application of a lotion treatment on the transparency film. The ABM, similar to real infant BM, fails cohesively, resulting in part of the ABM remaining on the transparency film and part of the ABM being removed. The more efficient the lotion composition is, the lower is the percentage of residual ABM on the transparency film.

The transparency film that is used is part #CEB00559 Transparency Film For Copiers, supplied by Corporate Express (Broomfield, Colo.). A sheet of transparency film is placed on a flat horizontal surface, such as a table or bench, and the film is anchored on the top and bottom to the flat surface using adhesive tape.

A template and a fine-tip marker are used to mark-off up to twelve 3 cm by 3 cm sites on individual sheets of the transparency film, i.e., up to twelve sites per sheet of film. For each lotion composition tested, three transparency film sites are treated with the lotion composition. Three sites also receive no anti-stick treatment, i.e., serves as a negative control. The locations of the various treatments, including the no-treatment sites, may be randomized among the sites on the transparency films. For each site that is treated, a predetermined amount of 200-400 μg/cm² of the lotion composition is applied in the center of the site with a powder-free finger cot, Catalog #56613-413 as available from VWR Scientific of West Chester, Pa. The applied lotion composition is then spread over the entire site (the boundary of which is defined by the marks made using the template) using the powder-free finger cot, by placing the finger cot on top of the agent or lotion composition and lightly rubbing the finger cot over the skin surface using several side-to-side and up-and-down movements for a total elapsed time of 10 seconds. Examining the site from an oblique angle, the person conducting the test needs to ensure that a uniform film has been formed over the entire area of the site. The film is left exposed to air, untouched, for approximately 1 minute prior to proceeding with the subsequent steps.

A 1 ml syringe, such as Catalog #BD-309628 as available from VWR Scientific of West Chester, Pa., that has been filled with room temperature ABM and is devoid of air bubbles, is placed onto a tared four-place analytical balance. The weight is recorded. The syringe with ABM is held over the center of the test site on the transparency film, approximately 2 mm from the transparency film surface, and approximately 0.2 mL of ABM is dispensed onto the transparency film by pressing the plunger and by watching the gradations on the syringe. The ABM should form a uniform, compact mound in the center of the test site. The syringe is re-weighed on the analytical balance, and the weight is recorded. The quantity of ABM that was delivered to the transparency film is calculated by subtracting the second weight from the first.

A 4 cm×4 cm piece of weigh paper, Catalog #12578-201 as available from VWR Scientific of West Chester, Pa., is tared on the four place analytical balance, centered over the ABM mound on the transparency film test site, and gently lowered onto the ABM using forceps. The weigh paper must not be touched with fingertips, as this may transfer oils onto its surface. Next, a 500 g bottle-shaped weight, such as Catalog #12766-518 as available from VWR Scientific of West Chester, Pa., that exerts approximately 0.5 psi of downward force is placed over the weigh paper such that the mound of ABM under the weigh paper is approximately centered under the weight. After 30 seconds have elapsed, the 500 g weight is slowly lifted. Using a pair of forceps, the weigh paper is slowly and gently peeled from the test site. The forceps are placed at the lower right corner of the weigh paper, and the weigh paper is slowly peeled upwards in the direction of the upper left corner of the weigh paper. It should take approximately 1-2 seconds to remove the weigh paper. Once removed, the weigh paper is placed back onto the analytical balance that it was tared on, and the weight is recorded to determine the amount of ABM removed.

The above steps are repeated until all sites on the transparency films have been tested, i.e., the steps consisting of application of lotion composition, application of ABM, application of weigh paper, application of weight, and removal of weigh paper. For the no-treatment control, application of agent or lotion composition is omitted and ABM is applied directly to the transparency film. The weight percent (%) residual ABM left on the transparency film after treatment is calculated from the weight measurements according to the equation ((ABM Applied−ABM Removed)/ABM Applied)×100.

The mean value for residual ABM and standard error of the mean for each lotion composition is calculated. When the method is run correctly, the no treatment control typically yields a value between approximately 30% to 50% residual ABM. To ensure reproducible results, the Anti-Stick Screening Method should be run at a room temperature of 21° C.±2° C. and at a relative humidity of 30-50%.

### Preparation of Artificial Pasty Bowel Movement (ABM)

The following equipment is required:
an analytical balance accurate to ±0.001 g
a homogenizer capable of stirring the ingredients to homogeneity, such as an Ika Labortechnik™ T25 basic or equivalent as available from Ika-Werke GmbH and Co. KG of Staufen, Germany.
a homogenizer probe to be used with the homogenizer, such as Catalog #S25N 25F as available from Ika-Werke GmbH and Co. KG of Staufen, Germany.

The following reagents are required:
Feclone™ Powder #4, available from SiliClone Studio, Valley Forge, Pa., as Catalog Number Feclone BFPS-4.
Feclone™ Powder #6, available from SiliClone Studio, Valley Forge, Pa., as Catalog Number BFPS-6.
Feclone™ Powder #7, available from SiliClone Studio, Valley Forge, Pa., as Catalog Number BFPS-7.
Carbopol™ 981, available from Noveon, Cleveland, Ohio.
Deionized water.

The following quantities of the above reagents are required:

| Ingredient | Grams |
|---|---|
| Deionized water for | 78.78 |
| Carbopol ™ solution | |
| Feclone ™ powder #4 | 6.600 |
| Feclone ™ powder #6 | 6.600 |
| Feclone ™ powder #7 | 6.600 |
| Carbopol ™ 981 | 0.900 |

The procedure to prepare the ABM consists of the following steps:

### A. Preparation of Carbopol™ Solution

1. Weigh 78.78 g±0.01 g of deionized water in a 250 ml beaker.
2. Weigh 0.900 g±0.001 g of Carbopol™ on weigh paper.
3. Put beaker on a magnetic stirrer and set speed at 400 rpm.
4. Add Carbopol™ powder slowly to the water, over the span of about 5 minutes. While adding the Carbopol™, increase the stirring speed slowly to 600 rpm.
5. Once the Carbopol™ powder has been added to the water, cover the beaker and continue mixing at 600 rpm for 15 minutes. The Carbopol™ powder must be completely dispersed, i.e., a transparent gel without any agglomerates.
6. Set up a hot plate at 150° C. Place the Carbopol™ solution on the hot plate and continue mixing at 600 rpm until the solution is heated to 81° C. to 83° C.

### B. Preparation of ABM Mixture

1. Weigh 6.600 g±0.01 g each of Feclone powders #4, #6, and #7 into a beaker and mix well.
2. Using a T25 basic or equivalent homogenizer with a homogenizer probe, stir the Carbopol™ solution at 8000 rpm for about 30 seconds before proceeding with Step 3.
3. To the Carbopol™ solution that is being stirred, slowly add the Feclone™ powder mixture, about one quarter of the total at a time. Ensure that the Feclone™ powder mixture gets pulled through the homogenizer probe during addition, i.e., is thoroughly mixed into the pasty lotion composition that is forming. If necessary, use a spatula to facilitate incorporation of the Feclone™ powder mixture into the lotion composition.
4. After all of the Feclone™ powder mixture has been added, continue mixing with the homogenizer at 8000 rpm for an additional 5 minutes, using the spatula to push the pasty lotion composition towards the homogenizer probe. The lotion composition should be thoroughly mixed and appear homogeneous.

The finished ABM may be placed in a container, such as Catalog #14233-954 as available from VWR Scientific of West Chester, Pa., and stored in the refrigerator for up to 30 days. After 30 days, a new sample should be prepared for further experiments. The container must be tightly sealed to avoid drying out of the ABM. Prior to using the ABM in the Anti-Stick Screening Method, the ABM must be removed from the refrigerator and allowed to adjust back to room temperature. An easy way to accomplish this is to fill a 10 ml syringe, such as Catalog #BD301604 as available from VWR Scientific of West Chester, Pa., with cold ABM and then allow the syringe to equilibrate to room temperature on a counter top. Equilibration typically takes about 15 minutes. The 10 ml syringe can then be used to fill the 1 ml syringe described in the Anti-Stick Method.

### Onset of Crystallization by Differential Scanning Calorimetry Method

The crystallization onset temperature of a lotion is measured using a differential scanning calorimeter (DSC): TA Instruments Q2000 fitted with a refrigerated cooling accessory and autosampler (TA Instruments, New Castle, Del.) with nitrogen purge. Pre-weigh a DSC pan and matching lid and record the weight to the nearest 0.1 mg. Using a die slightly smaller than the diameter of the DSC pan, punch a sample composed of 2-4 layers of article topsheet from an area containing lotion. The sample weight should be 2.0±0.5 mg, containing at least 0.5 mg of lotion. Place the sample in the pre-weighed DSC pan and record the weight to ±0.1 mg. Using a crimping press (TA Instruments, Wilmington, Del.), carefully crimp the lid on the DSC pan assuring proper seal and no contamination on the outside of the pan. In like fashion, prepare an empty pan with lid containing no sample for use as a reference pan. Place both pans into the instrument's autosampler, the sample in a sample slot and the reference in the reference slot.

Program the DSC to ramp at 20° C./min. to 80° C. (or a temperature at least 20° C. higher than the lotion's melt temperature), then hold isothermal at that temperature for 15 min., followed by a ramp at 20° C./min. back to 25° C. Initiate a run and collect data for the heating and cooling cycle.

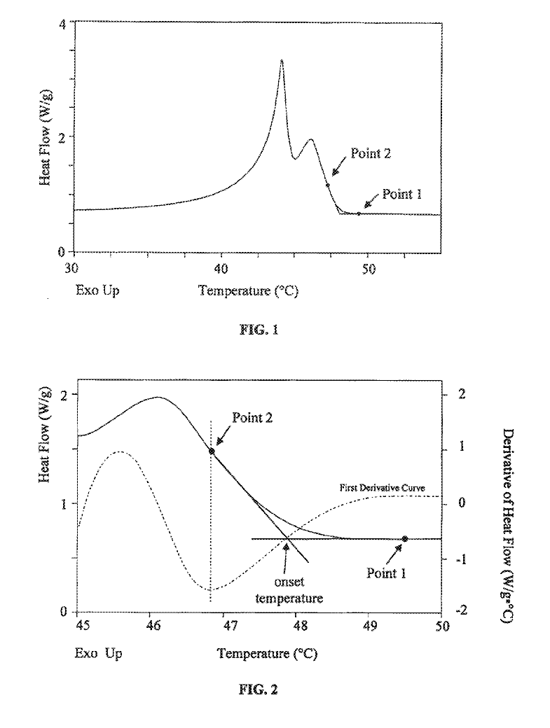

Calculate the onset temperature of the crystallization from the cooling data curve (i.e., second cycle). An example of a cooling data curve is shown in FIG. 1. Using the analysis software, overlay the curve of the heat flow and the curve of the first derivative of the heat flow with respect to temperature. FIG. 2 illustrates the overlaying of the two curves. With the onset function, select point 1 on the baseline of the heat flow curve before the beginning of the first peak. Select point 2 on the upward slope of the first heat flow peak, where the derivative heat flow gives the minimum associated with that peak. FIG. 2 illustrates an example of appropriate placement of the selected points. The software then draws tangents to the curve at the selected points to calculate the onset temperature.

Perform 3 replicates of the lotioned topsheet and report the crystallization onset temperature as the average to the nearest 0.1° C.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A substrate comprising a lotion composition, said lotion composition comprising:
a) from about 20 to about 80 weight percent of one or more compounds A which are liquid at 25° C., selected from the group consisting of liquid polyethylene glycol, liquid polyethylene glycol derivatives, liquid polypropylene glycol, liquid polypropylene glycol derivatives, liquid polyhydric alcohol, liquid fatty acid esters comprising at least one fatty acid unit and at least one ethylene glycol unit, liquid fatty acid esters comprising at least one fatty acid unit and at least one propylene glycol unit, and mixtures thereof;
b) from about 5 to about 50 weight percent of one or more compounds B which are solid at 25° C., selected from the group consisting of solid polyethylene glycol derivatives, solid polypropylene glycol derivatives, solid alkoxylated non-ionic surfactants, solid glycerol esters, solid sorbitan and derivatives, solid sucrose esters and their derivatives, solid glucose esters and their derivatives, and mixtures thereof;
c) from about 1 to about 40 weight percent of one or more crystallization accelerators C selected from the group consisting of C₁₄-C₂₂ fatty alcohols, C₁₂-C₂₂ fatty acids, solid fatty soap, waxes selected from the group consisting of carnauba, ozokerite, beeswax, candelilla, paraffin, ceresin, esparto, rezowax, isoparaffin, and mixtures thereof;
and wherein the weight ratio of compound(s) A to crystallization accelerator(s) C is from 3:2 to 10:1.

2. The substrate according to claim 1 wherein said lotion composition comprises:
a) from about 30 to about 70 weight percent of said one or more compounds A,
b) from about 10 to about 30 weight percent of said one or more compounds B,
c) from about 3 to about 25 weight percent of said one or more crystallization accelerators C.

3. The substrate according to claim 1 wherein said one or more compounds A are selected from the group consisting of liquid polyethylene glycol, liquid polyethylene glycol derivatives, liquid polypropylene glycol, liquid polypropylene glycol derivatives, liquid polyhydric alcohol and mixtures thereof.

4. The substrate according to claim 1 wherein said one or more compounds B are selected from the group consisting of solid polyethylene glycol derivatives, solid polyethylene glycol fatty alcohol ethers and mixtures thereof.

5. The substrate according to claim 1 wherein said one or more crystallization accelerators C are selected from the group consisting of C₁₄-C₂₂ fatty alcohols, C₁₂-C₂₂ fatty acids and mixtures thereof.

6. The substrate according to claim 1 wherein said lotion composition comprises a liquid polyethylene glycol as compound A, a polyethylene glycol fatty alcohol ether as compound B and a C₁₄-C₂₂ fatty alcohol as crystallization accelerator C.

7. The substrate according to claim 1 wherein said crystallization accelerator C is selected from the group consisting of cetyl alcohol, stearyl alcohol and mixtures thereof.

8. The substrate of claim 1 wherein the lotion composition further comprises at least one particulate material.

9. The substrate of claim 1 wherein said substrate is a nonwoven web.

10. An absorbent article comprising the substrate of claim 1.

11. The absorbent article of claim 10 wherein the absorbent article is selected from the group consisting of a diaper, a training pant, a feminine hygiene product, an adult incontinence product.

12. A wipe comprising the substrate of claim 1.

13. A process for manufacturing an absorbent article comprising a lotion composition limiting the adherence of feces or menses to the skin that comprises the steps of applying an effective amount of a lotion composition according to claim 1 from a melt thereof to the body facing surface of a substrate forming the topsheet of said absorbent article via spraying, printing, coating, extrusion or combination of these methods and resolidifying the lotion composition to form a solidified coating on the body facing surface of the substrate.

14. A method for reducing the adherence of feces or menses to the human skin comprising the steps of:
(i) providing a lotion composition comprising:
from about 20 to about 80 weight percent of one or more compounds A which are liquid at 25° C., selected from the group consisting of liquid polyethylene glycol, liquid polyethylene glycol derivatives, liquid polypropylene glycol, liquid polypropylene glycol derivatives, liquid polyhydric alcohol, liquid fatty acid esters comprising at least one fatty acid unit and at least one ethylene glycol unit, liquid fatty acid esters comprising at least one fatty acid unit and at least one propylene glycol unit, and mixtures thereof;
from about 5 to about 50 weight percent of one or more compounds B which are solid at 25° C., selected from the group consisting of solid polyethylene glycol derivatives, solid polypropylene glycol derivatives, solid alkoxylated non-ionic surfactants, solid glycerol esters, solid sorbitan and derivatives, solid sucrose esters and their derivatives, solid glucose esters and their derivatives, and mixtures thereof;
from about 1 to about 40 weight percent of one or more crystallization accelerators C selected from the group consisting of C₁₄-C₂₂ fatty alcohols, C₁₂-C₂₂ fatty acids, solid fatty soap, waxes selected from the group consisting of carnauba, ozokerite, beeswax, candelilla, paraffin, ceresin, esparto, rezowax, isoparaffin, and mixtures thereof;
and wherein the weight ratio of compound(s) A to crystallization accelerator(s) C is from 3:2 to 10:1;
(ii) applying an effective amount of said lotion composition to the body facing surface of a substrate used in the manufacture of, or as, or in an absorbent article, wherein the absorbent article is an article to be worn by a wearer;
(iii) contacting the skin of the wearer with said absorbent article.

15. A method for assessing the suitability of a lotion composition in a high speed manufacturing process for making an absorbent article, the method comprising determining the crystallization onset temperature of the lotion composition using the Differential Scanning calorimetry method as described herein.
